(19) United States
(12) Patent Application Publication
(10) Pub. No.: US 2014/0155897 A1
(43) Pub. Date: Jun. 5, 2014

(54) SURGICAL INSTRUMENT

(71) Applicants: Volker Heiny, Denzlingen (DE); Hans Uwe Lorius, Georgen (DE); Stefan Ohlig, Freiburg (DE)

(72) Inventors: Volker Heiny, Denzlingen (DE); Hans Uwe Lorius, Georgen (DE); Stefan Ohlig, Freiburg (DE)

(73) Assignee: RZ-MEDIZINTECHNIK GMBH, Tuttlingen (DE)

(21) Appl. No.: 13/879,651

(22) PCT Filed: Nov. 3, 2011
(86) PCT No.: PCT/EP2011/005544

§ 371 Date: Jun. 18, 2013

(30) Foreign Application Priority Data
Nov. 5, 2010 (DE) 10 2010 050 352.5

(51) Int. Cl. A61B 17/32; A61B 17/16

(52) U.S. Cl. CPC A61B 17/32002; A61B 17/162
USPC 606/79

(57) ABSTRACT

A surgical instrument for arthroscopic and surgical interventions, includes an elongate base body having an integrated drive unit via which a hollow-cylindrical drive element, which is rotatably mounted and arranged in an elongate, hollow outer shaft, is driven. The drive element is coupled at a distal end to a surgical cutting tool. The surgical instrument includes an exchangeable head as an adaptor. A disposable tube is arranged as part of a suctioning device.

### CROSS REFERENCE TO RELATED APPLICATIONS

This application relates to and claims priority from PCT/EP2011/005544 filed Nov. 3, 2011, the entire contents of which are incorporated herein by reference, which in turn claims priority formed Ser. No. 10 2010 050 352.5 filed Nov. 5, 2010.

### FIGURE SELECTED FOR PUBLICATION

FIG. 5

### BACKGROUND OF THE INVENTION

1. Field of the Invention

The present invention relates to a surgical instrument for arthroscopic and surgical intervention with a tool coupled at the distal end of a shaft to a drive element.

2. Description of the Related Art

Surgical instruments in the field are commonly designated by experts as shavers and customarily possess an elongate base body, in which is disposed a drive unit and a exchangeable head for a surgical cut-ting, abrading, or milling tool, wherein the tool is disposed at the distal-end portion of a hollow-cylindrical drive element, which pivots on an elongate, hollow, outer shaft. Such surgical instruments are as a rule combined with a suction arrangement, with the aid of which body fluid, blood, tissue, cartilage, or bone particles released during the operation, which might hamper the operational intervention, are suctioned off.

Besides instruments that are linearly constructed, surgical instruments are also known in which the area of the distal end is bent, in order to also be able to reach work positions that are difficult to access.

Surgical instruments of this type are described in numerous patents and patent applications, such as, for example, in EP 0 189 807 B1, EP 0 481 760 B1, and DE 44 14 903 A1, the entire contents of which are incorporated by reference.

In arthroscopy and arthroscopic operations, infection prophylaxis plays a large role, and the same basic hygienic prerequisites must be satisfied as in any aseptic operation on the large joints. So the surgical instruments used must be cleaned according to stringent hygienic guidelines in order to reduce the danger of contamination and infection.

Arthroscopic shavers exist in prior art for removing fine soft tissue or cartilage, as was already mentioned at the beginning, made of a base body with an integrated drive unit, onto which the tool is set, the so-called shaver blades, by means of a fitting. A coupling unit, disposed at the proximal end of a drive element, is introduced to this in effective connection with a corresponding coupling unit of the drive element integrated in the base body.

During surgical use, the chip and tissue remnants removed through the internal channel of the hollow-cylindrical drive element are suctioned off and are then directed further to a drain pan by means of a suction tube integrated into the base body and securely connected to it and disposed thereon. Because the shaver blades are replaceable and as a rule are used many times, the suction area of the surgical instrument, particularly, must be cleaned very carefully after each use, which leads to difficulties, in particular in the receptacle area for the shaver blade and in the area of the fitting connected directly to the outer hollow shaft, since these areas are difficult to access and usually exhibit many angles and edges, to which contamination can be taken up and be stuck. A further area especially in danger of contamination is the area where the suction tube enters the base body, which is particularly difficult to access. These circumstances lead to the fact that a sufficient infection prophylaxis is often only ensured if these areas undergo an additional endoscopic inspection. Since this additional check is extremely expensive and is preferably to be avoided, possible permanent danger consequently results from the areas cited above, so that germs formed there result in infections, which can have serious health consequences for the patient.

Accordingly, there is a need for an improved surgical instrument that addresses one of the concerns noted above.

### ASPECTS AND SUMMARY OF THE INVENTION

In response, it is now recognized that a surgical instrument is required that does not exhibit the drawbacks of the conventional constructions noted above.

The present invention relates to a surgical instrument for arthroscopic and surgical interventions, includes an elongate base body having an integrated drive unit via which a hollow-cylindrical drive element, which is rotatably mounted and arranged in an elongate, hollow outer shaft, is driven. The drive element is coupled at a distal end to a surgical cutting tool. The surgical instrument includes an exchangeable head as an adaptor. A disposable tube is arranged as part of a suctioning device.

The problem noted is solved by means of a surgical instrument with the features as discussed herein. Further developments and advantageous embodiments of the surgical instrument according to the invention are also noted herein.

The proposed invention provides an improvement for the hygienic conditions when using arthroscopic surgical instruments and to facilitate the cleaning of the instruments in a method for the same.

The problem is solved by means of a surgical instrument with a preferably elongate base body, into which a drive unit is integrated, by means of which a rotatably mounted, hollow-cylindrical drive element is driven, which is disposed in a hollow, outer, elongate shaft, which is on the other hand coupled at its distal-end portion to a surgical cutting, abrading, or milling tool. The base body includes, in addition, a control panel, a preferably ergonomic hand grip, a receptacle for a exchangeable head, and a cable connection.

Besides the base body, the surgical instrument according to the invention includes a exchangeable head as a fitting for the surgical cutting, abrading, and milling tool, a drive element disposed in an elongate, hollow, outer shaft and rotatably mounted thereon, and a cutting, abrading, or milling tool coupled to the drive element disposed on the distal-end portion of the disposed drive element, as well as a suction arrangement.

The essential idea according to the invention consists of using an exchangeable head for the surgical cutting, abrading, or milling tool, on which is removably disposed a one-way tube made of plastic, which is a part of the suction arrangement and which leads directly to the drain pan of the suction arrangement. Consequently, the suction tube, which is customarily integrated into the base body in surgical equipment of this type, is eliminated from the body and is replaced with the one-way tube mentioned above made of plastic, which is disposed directly on the exchangeable head and can be replaced anew after each operational intervention.

By means of this step, the especially critical area where the suction tube enters, which is hard to access, falls in the area of the base body in the receptacle for the exchangeable head for the tool as a potential path for the focus of infection, whereby a really essential step for improving infection prophylaxis can be achieved.

In order to create a suitable opportunity to attach the one-way tube to the base body, according to a preferred embodiment of the present invention, an ergonomic hand grip is disposed on the body, which can be swung down and which is provided as a guide and mount for the one-way tube. At the same time, the one-way tube lies on the inside of the ergonomic hand grip between the hand grip and the base body, whereby the disposition of the one-way tube on the outside of the base body is prevented, in an elegant manner, from impeding the operator in any way possible in his/her work.

A further advantageous embodiment of the ergonomic hand grip provides that it exhibit, at the same time, a clamping function for the exchangeable head, so that upon swinging the ergonomic hand grip up, the clamp on the exchangeable head is simultaneously released and conversely the exchangeable head is clamped on if the ergonomic hand grip lies against the base body.

A preferred embodiment of the present invention in addition provides that an air lever is disposed on the exchangeable head for regulating the suction air, which can be reached by the operator with the movement of a finger.

Starting from the replacement of the suction tube integrated into the base body with a one-way tube made of plastic, which leads directly to the exchangeable head, the hygienic concept can now be further completed in various ways.

In an advantageous embodiment, it is provided that the exchangeable head be a multipath exchangeable head, which essentially consists of an annular receptacle for the surgical cutting, abrading, or milling tool coupled to the drive element disposed in the outer shaft. In addition, integrated into the exchangeable head is an outlet support for the one-way tube, as well as an air lever, with which the suction air can be regulated. The annular, multipath exchangeable head is readily accessible and has preponderantly smooth surfaces on the inside, which can be simply and safely cleaned, so that the multipath exchangeable head can be re-used without increasing the risk of infection if the basic, requisite hygienic specifications are met.

A further, even safer variant provides that the exchangeable head be conceived as a one-way exchangeable head, in which the surgical cutting, abrading, or milling tool is fully integrated into the one-way exchangeable head with the drive element disposed in the outer shaft, and forms a unit with it. It is provided that the one-way exchangeable head together with the one-way tube be changed after use and disposed of The one-way exchangeable head, then, has the same functions as the multi-path exchangeable head, and it is automatically understood that a corresponding air lever is also disposed on the one-way exchangeable head to regulate suction air.

The manufacture of exchangeable heads occurs advantageously by means an injection-molding method from a thermoplastic and thermoset synthetic material, in which, in the case of a one-way exchangeable head, the surgical tool is extruded together with the hollow outer guide-shaft with a type of fitting that can be directly inserted together with the tool into the corresponding receptacle opening in the base body of the surgical instrument.

In a further advantageous embodiment of the surgical instrument according to the invention, a programmable electronic system with a keyboard disposed in the control panel is provided in the body for monitoring data relevant to operational safety. Preferably, the electronic system includes RFID electronics, which are combined with an RFID chip integrated into the surgical cutting, abrading, or milling tool. With the aid of this electronic system, the rpm can be adjusted and monitored, for example in real time. In addition, the operating time can be recorded in the RFID chip in the tool, so that tracking is possible as to how often and how long a specific tool is used. In this way, tools can be blocked from multiple usage in a simple manner, whereby the risk of infection can be reduced.

Recapitulating, it may be recorded that, according to the present invention, the risk of infection when using arthroscopic surgical instruments can be decisively reduced by means of various steps. What is more, in a first step, the principal focus of infection, namely the area where the suction tube enters the base body, is eliminated, in which a one-way tube is guided directly to the exchangeable head for the surgical cutting, abrading, or milling tool. When realizing the concept of using a multipath exchangeable head, the exchangeable head is, in addition, constructed so that it is easy to clean, whereby additional security against contamination is gained. In an especially advantageous embodiment, the exchangeable head is provided as a one-way exchangeable head, whereby contamination or infection elicited by the tool can be all but entirely avoided, because now all the areas presenting danger of contamination are eliminated and are provided for a one-time use only. Besides these steps, which concern the construction of the instrument, electronic monitoring is provided, which offers additional security and can prevent multiple usage of a tool, for example.

The above and other aspects, features and advantages of the present invention will become apparent from the following description read in conjunction with the accompanying drawings, in which like reference numerals designate the same elements.

### BRIEF DESCRIPTION OF THE DRAWINGS

FIGS. 1-3 provides in respective perspective views, provides the surgical instrument according to the invention with base body and exchangeable head in different stages of the assembly.

FIG. 4 provides a multipath exchangeable head in a plan view.

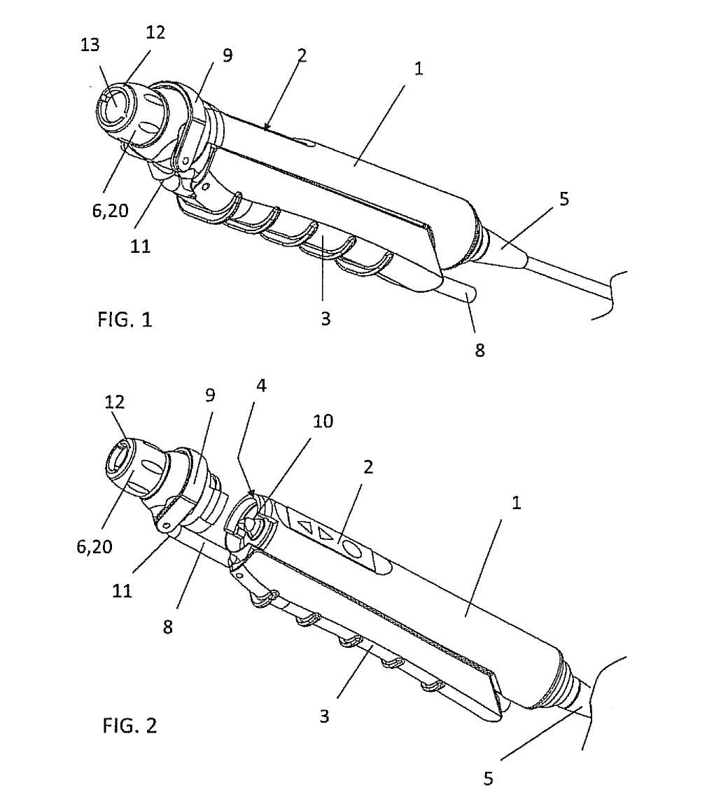

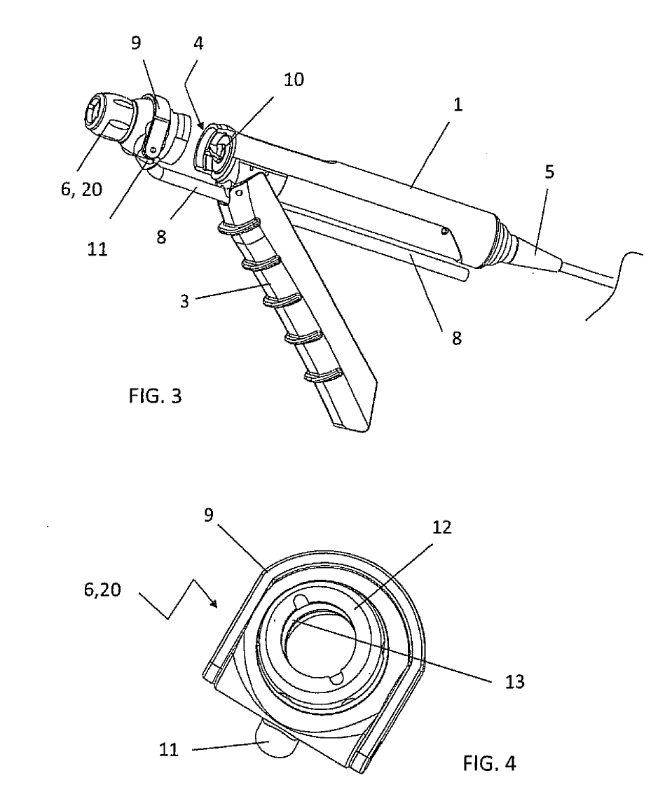

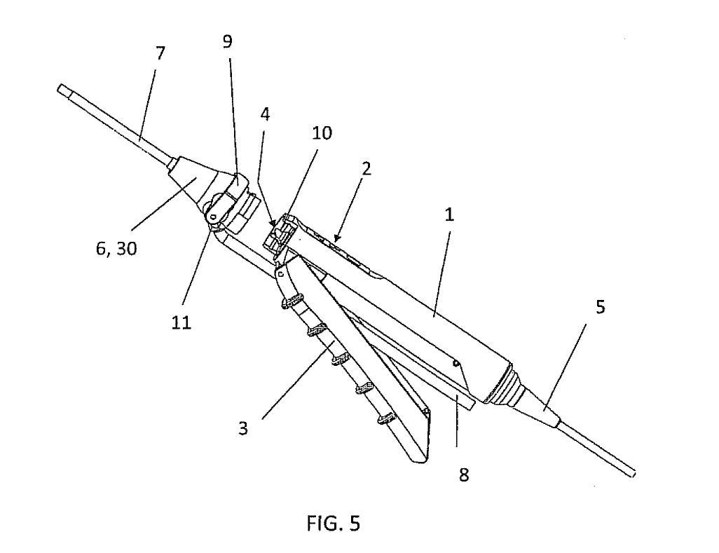

FIG. 5 provides the surgical instrument according to the invention in a perspective view with a one-way exchangeable head.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

Referring now to FIGS. 1-5 for review. FIG. 1 shows in a perspective representation the essential parts of the surgical instrument according to the invention. What is more, the elongate base body 1 may be recognized, into which the drive unit is integrated, which is, however, not depicted in this outside illustration. The base body 1 in addition includes a control panel 2, a cable connection 5, and an ergonomic hand grip 3. The exchangeable head 6 is a multi-path exchangeable head 20, which is mounted on the body 1 in the illustration selected for FIG. 1 and in the present case, is not fitted with a tool, so that one can look inside the annular receptacle 12 of the multipath exchange-able head 20, in which the smooth, easily cleaned internal surfaces 13 may be seen. The exchangeable head 6, 20 includes an outlet support 11, on which is mounted the one-way tube 8 made of plastic. The one-way tube 8 itself is freely movable, independent of the base body 1, and is incorporated into the ergonomic hand grip 3 which can be swung down, which is provided as a guide and mount for the one-way tube 8. In addition, in the area of the exchangeable head 6, 20 an air lever 9 may be seen, which is disposed within finger reach of the operator and with the aid of which the suction air can be regulated.

FIG. 2 shows the parts of the surgical instrument from FIG. 1, also in perspective representation, in which the exchangeable head 6, 20 of the base body I is now removed, so that a view is provided into the receptacle 4 for the exchangeable head 6, 20. What is more, it is clear that the receptacle is not very deep, is readily accessible, and therefore simple to clean. In the center of receptacle 4 is disposed a coupling unit 10, which is coupled to the drive unit located in the base body 1. Upon setting up the exchangeable head 6, 20 fitted with the tool, the coupling unit 10 acts in effective connection with a mating part disposed on the proximal end of the hollow cylindrical drive element, so that the surgical cutting, abrading, or milling tool, disposed at the distal-end portion of the drive element and coupled to the drive element, can be driven.

FIG. 3 corresponds in essence to FIG. 2, in which, in this case however, the ergonomic hand grip 3 is swung down. In this illustration, it may be seen that the one-way tube 8 made of plastic is totally independent of the base body 1 and is disposed loosely on the inside of the ergonomic hand grips 3, so that upon swinging the ergonomic hand grip 3 down, the multi- path exchangeable head 20 can be removed together with the one-way tube 8 with no problem.

FIG. 4 shows the multipath exchangeable head 20 in a perspective representation in plain view. The annular receptacle 12 of the multipath exchangeable head 20 is enclosed by the air lever 9, which is in effective connection with the outlet support 11 and with the aid of which the suction air can be regulated. This illustration clarifies the cleaning-friendly design and concept of the multipath exchangeable head 20, which is constructed as an annulus, in which the inside of the annular receptacle 12 is readily accessible, exhibits few corners or edges, and is accordingly easy to clean.

FIG. 5 now shows in a perspective view a complete arthroscopic surgical instrument with a one-way exchangeable head 30 in the disassembled state. The base body I with receptacle 4, coupling unit 10, control panel 2, ergonomic hand grip 3, and cable connection 5 is identical to the base body 1 from FIGS. 1-3 and can consequently as desired be used for a multipath exchangeable head 20 or a one-way exchangeable head 30. The dispositions on the one-way exchangeable head 30 are identical to those on the multipath exchangeable head 20. So the air lever 9 encloses the one-way exchangeable head 30 and is at the same time in effective connection with the outlet support 11 on which the one-way tube 8 is mounted. In this illustration, in addition, the elongate, hollow, outer shaft 7 may be seen, in which the hollow-cylindrical drive element is rotatably mounted, on the distal end of which is the cutting, abrading, or milling tool, while the proximal end includes the mating part for the coupling unit 10 on the base body 1.

By simply swinging the ergonomic hand grip 3 down, the exchangeable head 6, 20 is unclamped, so that the one-way exchangeable head can be removed and disposed of in a simple manner. The plan to use a one-way exchangeable head 30 is indeed more cost-intensive than using a multipath exchangeable head 20, but it has the advantage of offering an optimal infection prophylaxis, since the all the critical areas for germ formation or contamination are now eliminated. So ultimately, only the receptacle 4 on the base body I together with the coupling unit 10 still have to be cleaned, which is, on the one hand, very readily accessible and on the other hand results in few germs being in contact during an operational intervention.

### REFERENCE LIST

1 base body
2 control panel
3 ergonomic hand grip
4 receptacle (base body)
5 cable connection
6 exchangeable head
7 outer shaft
8 one-way tube
9 air lever
10 coupling unit
11 outlet support
12 receptacle (exchangeable head)
13 inner surface, exchangeable head
20 multipath exchangeable head
30 one-way replacement head

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

1-13. (canceled)

14. A surgical instrument system, comprising:
a base body with an integrated drive unit, an integrated operable control panel, a hand grip, a receptacle for an exchangeable head, and a cable connection;
said exchangeable head being operable for one of a surgical cutting, an abrading, and a milling;
a hollow-cylindrical rotatably mounted drive element operably disposed on an elongate, hollow, outer shaft;
a tool, being one of a surgical cutting tool, an abrading tool, or a milling tool, disposed on a distal-end portion of said drive element and operably coupled to the drive element; and
an integrated operative suction arrangement, further comprising:
a one-way operative tube made of plastic, which is operatively and removably disposed on the exchange-able head.

15. The surgical instrument system, according to claim 14, wherein:
said hand grip is disposed on the base body and is operative to be relatively swung down during a use thereof.

16. The surgical instrument system, according to claim 15, wherein:
said hand grip is operative as a guide and a mount for said one-way operative tube.

17. The surgical instrument system, according to claim 16, wherein:
said hand grip further comprises:
a means for operatively clamping said exchangeable head during said use.

18. The surgical instrument system, according to claim 17, further comprising:
an air lever, operative for regulating a suction air; and
said air lever disposed on said exchangeable head.

16. The surgical instrument system, according to claim 15, wherein:
said exchangeable head is a multipath exchangeable head; and
said exchangeable head further comprises:
an annular receptacle operative to receive said tool and operatively coupled to said drive element disposed on said outer shaft.

17. The surgical instrument system, according to claim 16, further comprising:
bounded inner surfaces on said annular receptacle; and
said inner surfaces being operatively smooth, thereby minimizing friction therewith.

18. The surgical instrument system, according to claim 14, wherein:
said exchangeable head is a one-way exchangeable head; and
said tool is operable integrated into said one-way exchangeable head and operably coupled with said drive element disposed on said outer shaft.

19. The surgical instrument system, according to claim 18, wherein:
said one-way exchangeable head is formed of one of a thermoplastic material and a thermoset synthetic material.

20. The surgical instrument system, according to claim 15, further comprising:
a programmable electronic system having a keyboard function feature;
said keyboard function feature disposed on said control panel;
said programmable electronic system disposed in said base body; and
said programmable electronic system operative to monitor an operational data feature during said use of said surgical instrument.

21. The surgical instrument system, according to claim 20, further comprising:
an RFID electronic component in said programmable electronics system; and
an RFID chip operably integrated into said tool and in an operative communication with said RFID electronic component during said use.

22. The surgical instrument system, according to claim 15, wherein:
said base body is an elongate base.

23. The surgical instrument system, according to claim 22, wherein:
said hand grip includes an ergonomically adapted portion for engaging a user's hand during said use.
